(19) United States
(12) Patent Application Publication
(10) Pub. No.: US 2016/0078782 A1
(43) Pub. Date: Mar. 17, 2016

(54) Glucose Ketone Index for MetabolicTherapy

(71) Applicants: Joshua J. Meidenbauer, Auburndale, MA (US); Thomas N. Seyfried, Foxboro, MA (US)

(72) Inventors: Joshua J. Meidenbauer, Auburndale, MA (US); Thomas N. Seyfried, Foxboro, MA (US)

(73) Assignee: THE TRUSTEES OF BOSTON COLLEGE, Chestnut Hill, MA (US)

(21) Appl. No.: 14/854,713

(22) Filed: Sep. 15, 2015

Related U.S. Application Data
(60) Provisional application No. 62/051,543, filed on Sep. 17, 2014.

(51) Int. Cl. G09B 19/00; A61B 5/00; G01N 33/49; A61B 5/145

(52) U.S. Cl. CPC G09B 19/0092; A61B 5/14532; A61B 5/14546; A61B 5/4866; A61B 5/486; G01N 33/492

(57) ABSTRACT

The ratio of blood glucose to blood ketones as a single Glucose Ketone Index value is tracked to manage metabolic treatment. This ratio identifies a metabolic state of health and has potential use for monitoring the progression of a metabolic or inflammatory disease or indication for all types of cancer, neurological disorders, and chronic inflammatory diseases. The tracking can be performed by a device or kit, such as a Glucose Ketone Index Calculator.

### CROSS REFERENCE

This application claims the benefit of the filing date of U.S. Provisional Patent Application Ser. No. 62/051,543, filed Sep. 17, 2014, which is hereby incorporated by reference in its entirety.

### STATEMENT OF GOVERNMENT SUPPORT

The current technology was developed in part using funds supplied by the National Institutes of Health (NIH) under grant No. NS055195. Accordingly, the U.S. Government has certain rights to this invention.

### FIELD OF THE INVENTION

The present invention is directed to the Glucose Ketone Index, a method for managing the metabolic treatment of a subject that tracks the Glucose Ketone Index, and a device useful therewith.

### BACKGROUND OF THE INVENTION

Chronic inflammatory disease is emerging as a major health crisis in the US. Many Americans are suffering from a plethora of chronic inflammatory diseases including obesity, Type 2 diabetes, cardiovascular disease, and cancer. Chronic and acute inflammation is also linked to several brain diseases to include epilepsy, Alzheimer's disease, Parkinson's disease, traumatic brain injury, autism, and glioma. Diet and lifestyle issues contribute to the pathology of those suffering from these and other inflammatory diseases. Hence, the management of inflammation is one means of reducing the health burden from these diseases.

Humans and other mammals have evolved to function for considerable periods without food. While glucose is the primary fuel for the brain under normal fed states, the water-soluble ketone bodies, beta-hydroxybutyrate (β-OHB) and acetoacetate, can compensate for glucose as a metabolic fuel for brain and other organs under conditions of prolonged fasting. In addition, acetone is a non-enzymatic metabolic byproduct of acetoacetate, and its presence in the breath of individuals can be used to estimate blood ketone values. The liver makes ketone bodies naturally from fatty acids released largely from stored body fat (triglycerides). Changes in key hormones, insulin and glucagon, and regulatory genes orchestrate the physiological transition between the glucose-dependent fed state and the lipid-dependent fasted state. Although ketoacidosis was linked originally to the pathology of diabetes, physiological ketosis is generally linked to improved health. The difference in blood β-OHB levels distinguishes ketoacidosis (>20 mM) from physiological ketosis (1-8 mM). Acetone is a non-enzymatic degradation product of acetoacetate that can be easily detected in the breath of individuals with diabetic ketoacidosis (mostly type 1), or in those who conduct prolonged water-only therapeutic fasting.

Calorie restriction (CR) is a type of metabolic therapy that has long been recognized as a means to improve general health, to manage a broad range of chronic diseases, and to delay aging. Chronic inflammatory diseases produce oxidative stress that ultimately damage tissue biomolecules. Hyperglycemia can contribute to oxidative stress and inflammation. Oxidative stress accelerates entropy, the thermodynamic basis for the aging process. CR reduces oxidative stress, which can delay entropy. The therapeutic effects of CR are associated with the reduction of blood glucose and the elevation of blood ketone bodies within normal physiological ranges. It has been shown that metabolism of ketone bodies can reduce oxidative stress by increasing the redox span of the mitochondrial Coenzyme Q couple, which reduces the amount of the Q semiquinone and thus oxygen radical production in cells with normally functioning mitochondria. It has been difficult to determine, however, if the major health benefits of CR are related to the elevation of ketone bodies, the reduction of glucose, or to the unique metabolic state arising from the combination of these effects.

Although periodic water-only fasting can improve health, prolonged fasting will eventually lead to the pathological state of starvation, while prolonged CR can lead to nutritional imbalances unless the nutritional composition of the restricted diet is carefully monitored. Nevertheless, emerging evidence indicates that dietary therapies, which lower glucose and elevate ketone bodies, are safe in children and adults, and are effective for a variety of metabolic, neurological and neurodegenerative diseases. It was recently shown that blood glucose is a major predictor of body weight in the C57/BL6 mouse strain. CR lowers blood glucose and body weight while elevating ketones regardless of the macronutrient composition of the consumed diet. Most popular weight loss diets are a form of CR.

A ketogenic diet (KD) is a low carbohydrate high-fat diet that was designed originally to manage seizures in children with epilepsy. The KD mimics the physiological state of fasting, which was known since the time of Hippocrates to reduce seizure susceptibility. An energy transition from carbohydrate metabolism to fat metabolism is thought to contribute to the therapeutic benefits of KDs. As with therapeutic fasting and CR, it remains unclear if the anti-epileptic and anti-convulsant effects of the KD are due to reduced glucose, to elevated ketone bodies, or to some combination of these effects. Circulating ketone levels become higher when KDs are administered in restricted amounts than when administered in unrestricted amounts. It was recently described how restricted KDs, administered with drugs and hyperbaric oxygen therapy, could help manage cancer. A similar therapeutic strategy could be used for managing cancer, neurological, and neurodegenerative diseases.

The KD is a metabolic therapy that must be administered with care, as would be the case for any medical therapy. It should be recognized that unrestricted or excessive consumption of KDs, which elevate body weight, could potentially produce hyperlipidemia and insulin insensitivity thereby reducing therapeutic benefit. The high fat concentration of the KD will usually prevent excess consumption and body weight gain. As with CR, however, evidence indicates that KDs and ketone bodies can have powerful therapeutic benefit for a broad range of metabolic, neurological, and neurodegenerative diseases, to include cancer. Recent evidence indicates that the therapeutic effects of β-OHB against oxidative stress can arise through its action as an endogenous histone deacetylase inhibitor. These findings reveal how a global shift in energy metabolism from glucose to ketone bodies can regulate gene expression through epigenetic mechanisms. Synthetic ketone esters also appear to replicate several therapeutic features of KDs, but further studies will be necessary to establish these connections. The molecular mechanisms by which ketone bodies underlie therapy are now under active investigation. The ketogenic diet could be an alternative or complementary metabolic therapeutic strategy for managing malignant brain cancer.

Prognosis remains poor for metastatic and invasive cancer in both children and adults. Although genetic heterogeneity is extensive in most cancers, the Warburg effect (aerobic fermentation of glucose) is a common metabolic malady expressed in nearly all neoplastic cells, regardless of tissue origin. Aerobic fermentation is necessary to compensate for the insufficiency of mitochondrial oxidative phosphorylation in the cells of most tumors. Indeed, mitochondrial structure and function is abnormal in malignant gliomas from both mice and humans. Normal brain cells gradually transition from the metabolism of glucose to the metabolism of ketone bodies (primarily β-hydroxybutyrate and acetoacetate) for energy when circulating glucose levels become limiting. Ketone bodies are derived from fatty acids in the liver and are produced to compensate for glucose depletion during periods of food restriction. Ketone bodies bypass the glycolytic pathway in the cytoplasm and are metabolized directly to acetyl CoA in the mitochondria. Tumor cells are less capable than normal cells in metabolizing ketone bodies for energy due to their mitochondrial defects.

Metabolic therapy using KDs is emerging as an alternative or complementary approach to the current standard of care for cancer management. This therapeutic strategy targets the aerobic fermentation of glucose (Warburg effect), which is the common metabolic malady of most tumors. The KD targets tumor energy metabolism by lowering blood glucose and elevating blood ketones (β-hydroxybutyrate). Tumor cells, unlike normal cells, cannot use ketone bodies effectively for energy when glucose becomes limiting. Although plasma levels of glucose and ketone bodies have been used alone to predict the therapeutic success of metabolic therapy, daily glucose levels can fluctuate widely in cancer patients. This can create difficulty in linking changes in blood glucose and ketones to efficacy of metabolic therapy.

### SUMMARY OF THE INVENTION

It is an aspect of the present invention to provide a method for managing the metabolic treatment of a subject including administering a diet to the subject, wherein the diet reduces blood glucose levels and elevates blood ketone levels in the subject; tracking the ratio of blood glucose to blood ketone levels in the subject as a single Glucose Ketone Index value; and maintaining the tracked Glucose Ketone Index value within a target range.

It is an aspect of the present invention to provide a device or kit capable of computing the ratio of blood glucose to blood ketones as a single Glucose Ketone Index value.

The current invention demonstrates features and advantages that will become apparent to one of ordinary skill in the art upon reading the attached Detailed Description.

### BRIEF DESCRIPTION OF THE DRAWINGS

FIG. 1 is a graph conceptually illustrating the relationship of sample plasma glucose and ketone body levels to cancer management in accordance with an embodiment of the present invention;

FIG. 2 is a graph plotting the sample glucose and ketone values from FIG. 1 against a metabolic zone of management of tumors in accordance with an embodiment of the present invention;

FIG. 3 is a graph plotting the Glucose Ketone Index sample values from Table 2 of a fictional patient over the course of one month in accordance with an embodiment of the present invention; and

FIG. 4 is a flow chart illustrating the operation of the GKIC device in accordance with an embodiment of the present invention.

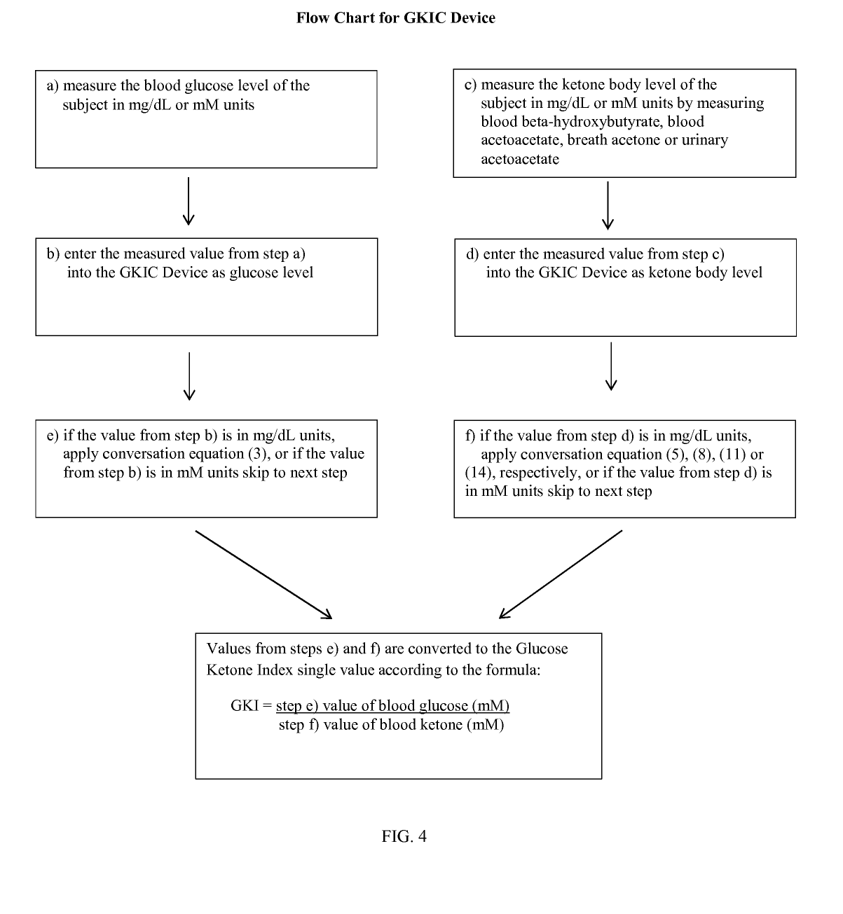

### DETAILED DESCRIPTION OF INVENTION

The present disclosure relates to a method for managing the metabolic treatment of a subject including administering a diet to the subject; tracking the ratio of blood glucose to blood ketones in the subject as a single Glucose Ketone Index value; and maintaining the tracked Glucose Ketone Index value within a target range.

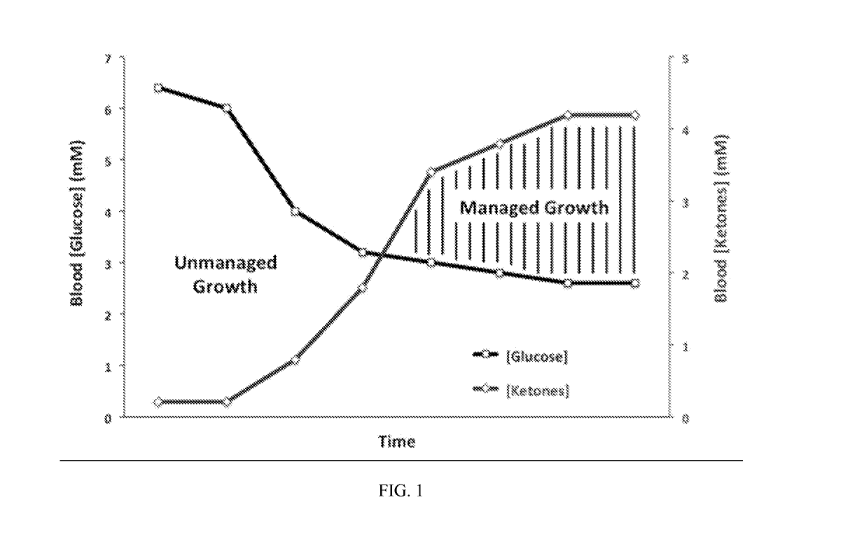

Therapies that can lower glucose and elevate ketone bodies will place more energy stress on the tumor cells than on the normal cells, including brain cells. This therapeutic strategy is illustrated conceptually with sample values shown in FIG. 1. As can be seen, as glucose falls and ketones rise, an individual enters the metabolic zone of management. This can be tracked utilizing the Glucose Ketone Index (GKI), which tracks the ratio of blood glucose to ketones as a single value. In FIG. 1, the glucose and ketone (β-OHB) values are shown within normal physiological ranges under fasting conditions in humans. This state is referred to as the zone of disease management. The glucose and ketone levels predicted for disease management are approximately 2.9-3.8 mM and 2.5-10.0 mM, respectively. Blood glucose levels can be lowered and ketone levels elevated to a greater degree in humans than in mice under calorie restriction and fasting. This difference is due to the larger brain size and the lower basal metabolic rate in humans than in mice. Consequently, the therapeutic benefits of CR and KDs could be greater in humans than in mice. However, daily activities and emotional stress can cause blood glucose levels to vary making it difficult for some people to enter the predicted therapeutic zone of management when tracking blood glucose levels alone.

Thus, the present invention provides a stable measure of systemic energy metabolism to predict management of metabolic, neurological, and neurodegenerative diseases, to include cancer. The ratio of blood glucose to blood ketone bodies, such as β-hydroxybutyrate (β-OHB) provides a better indication of metabolic management than could measurement or tracking of either blood glucose or ketone body levels alone.

The present invention calculates a ratio of blood glucose to blood ketones as a single value to manage metabolic treatment. The tracking can be performed by the Glucose Ketone Index Calculator (GKIC) and the ratio calculated is termed the Glucose Ketone Index (GKI). The GKIC can help assess the efficacy of metabolic therapy in animal models and patients with malignant brain tumors and with other cancers that express aerobic fermentation. The GKI prognosticates the success of metabolic therapy in managing disease and disorders. The GKI allows the user to adjust their diet and the implementation of metabolic therapy to maximize ketosis (preferred method of metabolism to manage disease) and minimize glucose metabolism. The GKIC allows the user to interpret their metabolic state and make necessary dietary adjustments to alter their metabolic state.

The GKIC can be used to compute the GKI for data published on blood glucose and ketone levels in humans and mice with brain tumors, as shown in Table 1 below. The results show a clear relationship between the GKI and therapeutic efficacy using ketogenic diets and calorie restriction. The GKI ratio identifies a metabolic state of health. The therapeutic efficacy of metabolic management through the use of dietary therapy, nutritional supplementation, or pharmacological therapy is greater with lower GKI values than with higher values. The GKI ratio has potential use for monitoring the progression of a metabolic or inflammatory disease or indication. The current invention has potential indications: for all types of cancer, brain cancer in particular; neurological disorders including epilepsy, Alzheimer's disease, Parkinson's disease, autism, and traumatic brain injury; chronic inflammatory diseases including obesity, Type 2 diabetes, and cardiovascular disease. The present invention also includes a device, with related Glucose Ketone Index Calculator (GKIC) built in, containing software for measuring the level of the blood glucose and blood ketones and generating a single GKI value.

The calculation of GKI is meant to include any method that can estimate blood glucose and blood beta-hydroxybutyrate (ketone) values. It may be possible to measure ketones in saliva or sweat in the future.

The Glucose Ketone Index (GKI) is capable of tracking the zone of metabolic management for brain tumor management. The GKI is a biomarker that refers to the molar ratio of circulating glucose over β-OHB, which is the major circulating ketone body. A mathematical tool called the Glucose Ketone Index Calculator was developed that can calculate the GKI and monitor changes in this parameter on a daily basis (Equation 1). The GKIC generates a single value that can assess the relationship of the major fermentable tumor fuel (glucose) to the non-fermentable fuel (ketone bodies). Because many commercial blood glucose monitors give outputs in mg/dL, rather than millimolar (mM), the GKIC is capable of converting the units to millimolar (Equation 1). In an embodiment, the kit can include a blood glucose monitor, blood ketone monitor, and a GKIC device. Included in the software program of the GKIC is a unit converter for both glucose and ketones (β-OHB), which can convert glucose and ketone values from mg/dL to mM and from mM to mg/dL (Equations 2, 3, 4, and 5) as necessary. The molecular weights used for calculations in the GKIC are 180.16 g/mol for glucose and 104.1 g/mol for β-OHB, which is the major circulating ketone body and that measured in most commercial testing kits. The unit converter allows for compatibility for a variety of glucose and ketone testing monitors.

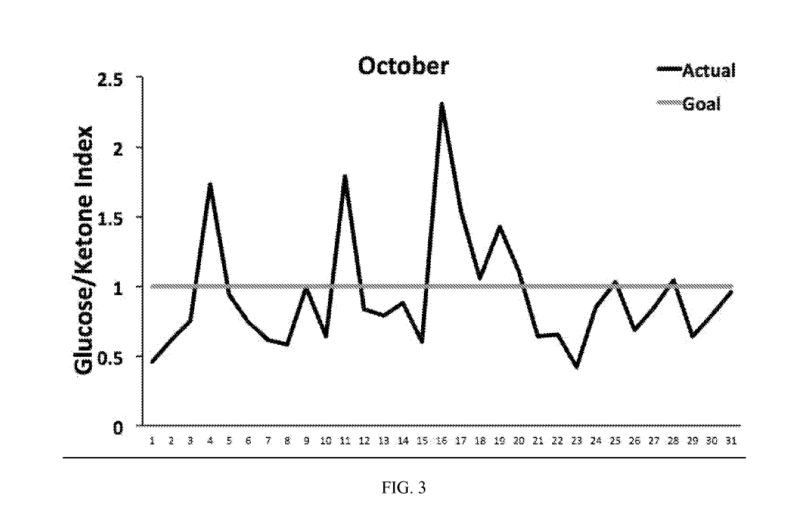

The GKIC can set a target GKI value to help track therapeutic status. Daily GKI values can be plotted to allow visual tracking of progress against an initial index value over monthly periods. Entrance into the zone of metabolic management would be seen as the GKI value falls below the set target value (as illustrated in FIG. 3). Additionally, the GKIC can track the number of days that an individual falls within the predicted target zone.

Suitable GKI target value ranges in accordance with embodiments of the present invention include from about 0.5 to about 5; about 0.5 to about 3; and about 0.5 to about 1.

Evidence is presented showing that the GKI can predict success for brain cancer management in humans and animals using metabolic therapies that lower blood glucose and elevate blood glucose levels. Besides ketogenic diets, calorie restriction, low carbohydrate diets, ketone ester supplementation, and therapeutic fasting can also lower blood glucose and elevate β-OHB levels and can have anti-tumor effects. The GKIC was developed to more reliably and simply predict therapeutic management for cancer patients under these dietary states than could measurements of either blood glucose or ketones alone. The data presented in Table 1 support this prediction. As tumor cells are dependent on glucose for survival and cannot effectively use ketone bodies as an alternative fuel, a zone of metabolic management can be achieved under conditions of low glucose and elevated ketones. Ketone bodies also prevent neurological issues associated with hypoglycemia, which allows blood glucose levels to be lowered even further. Hence, ketone body metabolism can protect normal brain cells under conditions that target tumor cells. The zone of metabolic management is considered the therapeutic state that places maximal metabolic stress on tumor cells while protecting normal cells.

The zone of metabolic management in an embodiment is likely entered with GKI values between 1.0 and 2.0 for humans. The Glucose Ketone Index can track the metabolic zone of glioma management. Therapeutic efficacy is considered best with index values of 1.0 or below.

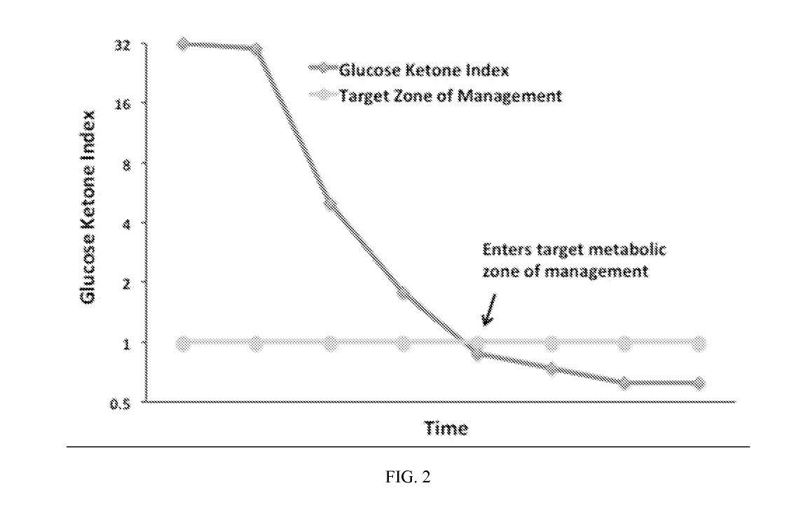

Optimal management is predicted for values as close to 1.0 as possible, or below. To illustrate the utility that the GKI serves for tracking an individual's progress, FIG. 2 shows the sample glucose and ketone values from FIG. 1 plotted against the individual's personal target metabolic zone of management. When an individual's GKI falls below the line denoting the target metabolic state, the zone of management is achieved.

There are alternative ways to calculate the Glucose Ketone Index (GKI) using ketone bodies other than beta-hydroxybutyrate (β-OHB). These include blood acetoacetate, breath acetone, and urinary acetoacetate. Any of the four methods using the described ketone bodies to calculate the GKI may be used alone or in combination.

$$\begin{matrix}[ Glucose Ketone Index ] = \frac{[ Glucose ( mg / dL ) ] / 18.016}{[ β \text{-} OHB ( mM ) ]} & ( 1 ) \\ [ Glucose ( mg / dL ) ] = [ Glucose ( mM ) ] \times 18.016 & ( 2 ) \\ [ Glucose ( mM ) ] = \frac{[ Glucose ( mg / dL ) ]}{18.016} & ( 3 ) \\ [ β \text{-} OHB ( mg / dL ) ] = [ β \text{-} OHB ( mM ) ] \times 10.41 & ( 4 ) \\ [ β \text{-} OHB ( mM ) ] = \frac{[ β \text{-} OHB ( mg / dL ) ]}{10.41} & ( 5 )\end{matrix}$$

Acetoacetate is a circulating ketone that is found in the blood. Acetoacetate is measured at half the number of beta-hydroxybutyrate molecules present in the blood. To measure the Glucose Ketone Index using blood acetoacetate values in place of beta-hydroxybutyrate values, the molarity of 102.088 g/mol is used for acetoacetate. The following calculations (Equations 6-8) can be used to calculate the Glucose Ketone Index from Acetoacetate values:

$$\begin{matrix}[ Glucose Ketone Index ] = \frac{[ Glucose ( mg / dL ) ] / 18.016}{2 * [ Acetoacetate ( mM ) ]} & ( 6 ) \\ [ Acetoacetate ( mg / dL ) ] = [ Acetoacetate ( mM ) ] \times 10.2088 & ( 7 ) \\ [ Acetoacetate ( mM ) ] = \frac{[ Acetoacetate ( mg / dL ) ]}{10.2088} & ( 8 )\end{matrix}$$

Acetone is a volatile ketone body that is present in the breath of individuals. The molarity of acetone is 58.08 g/mol. For devices that measure acetone values in the breath as a proxy for ketone bodies, acetone can be used in the following calculations (Equations 9-11) to calculate the Glucose Ketone Index:

$$\begin{matrix}[ Glucose Ketone Index ] = \frac{[ Glucose ( mg / dL ) ] / 18.016}{0.11 * {[ Acetone ( mM ) ]}^{0.47} - 0.18} & ( 9 ) \\ [ Acetone ( mg / dL ) ] = [ Acetone ( mM ) ] \times 5.808 & ( 10 ) \\ [ Acetone ( mM ) ] = \frac{[ Acetone ( mg / dL ) ]}{5.808} & ( 11 )\end{matrix}$$

Urinary acetoacetate can be used as a proxy for blood beta-hydroxybutyrate. For devices that measure urinary acetoacetate as a proxy for ketone bodies, the following calculations (Equations 12-14) can be used to calculate the Glucose Ketone Index:

$$\begin{matrix}[ Glucose Ketone Index ] = \frac{[ Glucose ( mg / dL ) ] / 18.016}{0.45 * {[ Urinary Acetoacetate ( mM ) ]}^{0.5} + 0.1} & ( 12 ) \\ [ Urinary Acetoacetate ( mg / dL ) ] = [ Urinary Acetoacetate ( mM ) ] \times 10.2088 & ( 13 ) \\ [ Urinary Acetoacetate ( mM ) ] = \frac{[ Urinary Acetoacetate ( mg / dL ) ]}{10.2088} & ( 14 )\end{matrix}$$

The above methods include the calculation of the GKI through any method that measures glucose and/or the three ketone bodies through the fluids of blood, urine, and breath. This includes through enzymatic reactions and biochemical analyses, to include but not limited to the noninvasive biomonitoring techniques of bioimpedance spectroscopy, electromagnetic sensing, fluorescence technology, mid-infrared spectroscopy, near infrared spectroscopy, optical coherence tomography, optical polarimetry, Raman spectroscopy, reverse iontophoresis, and ultrasound technology. Noninvasive biomonitoring techniques includes any technique that does not involve pricking (breaking) the skin.

The current noninvasive techniques being developed for glucose monitoring, including the current device, may be incorporated into smartphones, smartwatches, or other personal devices to measure glucose.

The GKIC could have utility not only for management of brain cancer and possibly other cancers dependent on glucose and aerobic fermentation for survival, but also for other diseases or conditions where the ratio of glucose to ketone bodies could be therapeutic. Such disease and conditions include Alzheimer's disease, Parkinson's disease, traumatic brain injury, migraines, epilepsy, and autism. Indeed, the ketogenic diet has long been recognized as an effective therapeutic strategy for managing refractory seizures in children. Therapeutic success in managing generalized idiopathic epilepsy in EL mice can also be seen when applying the GKI to the data presented on glucose and β-OHB. Further studies will be needed to determine the utility of the GKIC for predicting therapeutic success in the metabolic management of disease.

The GKI predicts success by providing real-time data on the metabolic state of the user. Sustained ketosis as a mode of metabolism is central to metabolically managing disease, and the GKI provides actionable data that can be used to alter dietary therapy to maximize therapeutic success. The GKI tracks the efficacy of the implementation of the diet, and allows the user to make dietary adjustments to shift their metabolic state to reach a targeted GKI. The GKI can be used to track and correlate index values to establish target zones to successfully manage a variety of metabolic diseases. In an embodiment, the GKI can be lowered through further calorie restriction and/or a reduction in the intake of glucogenic compounds, e.g., carbohydrates and glucogenic amino acids. In an embodiment, the GKI can be increased through increasing calorie intake and/or the addition of glucogenic compounds, e.g., carbohydrates and glucogenic amino acids.

The Glucose Ketone Index Calculator can calculate the GKI and monitor changes in this parameter on a daily basis (Equation 1). Table A below shows a sample calculation.

TABLE A
Instructions: Enter your blood glucose values (in mg/dL) and blood ketone
(β-hydroxybutyrate) levels (in mmol) into the respective boxes to calculate
your glucose/ketone index. Enter your desired glucose/ketone index value
to help you keep track of your progress.

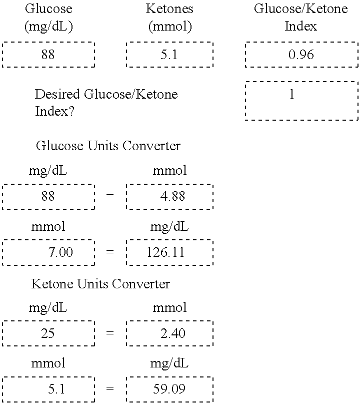

The GKIC can set a target GKI value to help track therapeutic status. Daily GKI values can be plotted to allow visual tracking of progress against an initial index value over monthly periods. Table B below shows sample values and FIG. 3 shows a plot of the sample values.

TABLE B
October

| | Glucose | Ketones | Glucose/Ketone | Desired |
|---|---|---|---|---|
| Date | (mg/dL) | (mmol) | index | Level? |
| 1 | 61 | 7.4 | 0.46 | Yes! |
| 2 | 71 | 6.4 | 0.62 | Yes! |
| 3 | 80 | 5.9 | 0.75 | Yes! |
| 4 | 105 | 3.4 | 1.73 | No |
| 5 | 100 | 5.9 | 0.94 | Yes! |
| 6 | 83 | 6.2 | 0.74 | Yes! |
| 7 | 73 | 6.6 | 0.61 | Yes! |
| 8 | 69 | 6.6 | 0.58 | Yes! |
| 9 | 89 | 5 | 0.99 | Yes! |
| 10 | 76 | 6.6 | 0.64 | Yes! |
| 11 | 103 | 3.2 | 1.79 | No |
| 12 | 87 | 5.8 | 0.83 | Yes! |
| 13 | 70 | 4.9 | 0.79 | Yes! |
| 14 | 85 | 5.4 | 0.88 | Yes! |
| 15 | 68 | 6.3 | 0.60 | Yes! |
| 16 | 108 | 2.6 | 2.31 | No |
| 17 | 100 | 3.6 | 1.54 | No |
| 18 | 97 | 5.1 | 1.05 | No |
| 19 | 95 | 3.7 | 1.43 | No |
| 20 | 84 | 4.2 | 1.11 | No |
| 21 | 74 | 6.4 | 0.64 | Yes! |
| 22 | 78 | 6.6 | 0.65 | Yes! |
| 23 | 56 | 7.4 | 0.42 | Yes! |
| 24 | 96 | 6.3 | 0.85 | Yes! |
| 25 | 87 | 4.7 | 1.03 | No |
| 26 | 78 | 6.3 | 0.69 | Yes! |
| 27 | 112 | 7.4 | 0.84 | Yes! |
| 28 | 90 | 4.8 | 1.04 | No |
| 29 | 76 | 6.6 | 0.64 | Yes! |
| 30 | 84 | 5.9 | 0.79 | Yes! |
| 31 | 88 | 5.1 | 0.95 | Yes! |

Average

| | |
|---|---|
| Glucose | 84.68 |
| Ketone | 5.56 |
| GK Index | 0.93 |

Low

| | |
|---|---|
| Glucose | 56 |
| Ketone | 2.6 |
| GK Index | 0.42 |

High

| | |
|---|---|
| Glucose | 112 |
| Ketone | 7.4 |
| GK Index | 2.31 |

Range

| | |
|---|---|
| Glucose | 56.00 |
| Ketone | 4.80 |
| GK Index | 1.89 |

Time in Metabolic
Zone of Management

| | |
|---|---|
| Yes | 71.0% |
| No | 29.0% |

Entrance into the predicted zone of metabolic management would be seen as the GKI value falls below the set target value (as illustrated in FIG. 2). The GKIC can track progress towards the predicted GKI target value needed for metabolic management of brain tumors. GKIC tracks an individual entering their target metabolic zone of management. Tumor progression is predicted to be slower within the metabolic target zone than outside of the zone.

The following examples are provided to illustrate embodiments of the present invention but are by no means intended to limit its scope. The examples described herein will be understood by one of ordinary skill in the art as exemplary protocols. One of ordinary skill in the art will be able to modify the below procedures appropriately and as necessary.

Without further elaboration, it is believed that one skilled in the art can, based on the above description, utilize the present invention to its fullest extent. The following specific embodiments and examples are, therefore, to be construed as merely illustrative, and not limitative of the remainder of the disclosure in any way whatsoever.

### Example 1

The GKIC was used to estimate the GKI for humans and mice with brain tumors that were treated with either calorie restriction or ketogenic diets from five previously published reports (Table 1). The results show a clear relationship between the GKI and efficacy of metabolic therapy using either the KD or calorie restriction. Table 1 shows low glucose ketone index values are correlated to improved prognoses in humans and mice with brain tumors. The therapeutic efficacy of the KD or calorie restriction is greater with lower GKI values than with higher values.

TABLE 1
Low Glucose Ketone Index values are correlated to improved prognoses in humans and mice with brain tumors.

| | | | # of | Days | Glu- | Ke- | Glucose | |
|---|---|---|---|---|---|---|---|---|
| | | | Sub- | on | cose | tones^{f} | Ketone | |
| Subjects | Tumor Type | Diet | jects | Diet | (mM) | (mM) | Index | Prognosis |
| Human⁴² | Anaplastic Astrocytoma | KD-UR^{a} | 1 | 0 | 5.5 | 0.2 | 27.5 | No response to standard chemotherapy |
| | (Stage IV) | | | 56 | 5.0 | 4.6 | 1.1 | FDG uptake at tumor site was decreased by |
| | | | | | | | | 21.77%; tumor margins were unchanged |
| | Cerebellar Astrocytoma | KD-UR | 1 | 0 | 5.5 | 0.2 | 27.5 | Tumor resected and initiated on KD while |
| | (Grade III) | | | | | | | under standard chemotherapy, after tumor |
| | | | | | | | | was radiologically stable by CT |
| | | | | 56 | 4.0 | 5.5 | 0.7 | FDG uptake at tumor site was decreased by 21.84% |
| | | | | | | | | Notes: Both patients remained in remission after |
| | | | | | | | | return to standard diet for 5 years (Subject 1) |
| | | | | | | | | and 4 years (Subject 2), at time of publication |
| Human⁴³ | Glioblastoma | KD-R^{b} | 1 | 0 | 7.5 | 0.2^{g} | 37.5 | Incomplete surgical resection of tumor; received |
| | Multiforme (Grade IV) | | | | | | | chemotherapy and radiation therapy concurrent |
| | | | | | | | | with diet |
| | | | | 21 | 3.5 | 2.5^{g} | 1.4 | No evidence of tumor by MRI after concurrent therapy |
| | | | | | | | | Notes: Patient stayed on low calorie diet for an |
| | | | | | | | | additional 5 months; tumor recurrence 3 months after |
| | | | | | | | | low-calorie diet suspension |
| Mouse³³ | mouse CT-2A astrocytoma | SD-UR^{c} | 7 | 13 | 9.1 | 0.6 | 15.2 | Tumor dry weight: 55 ± 15 mg^{h} |
| | syngenic (C57BL/6J) | SD-R^{d} | 6 | 13 | 5.2 | 1.4 | 3.7 | Tumor dry weight: 7 ± 7 mg |
| | | KD-UR | 14 | 13 | 11.4 | 1.0 | 11.4 | Tumor dry weight: 70 ± 15 mg |
| | | KD-R | 6 | 13 | 5.7 | 1.3 | 4.4 | Tumor dry weight: 14 ± 8 mg |
| Mouse⁴⁴ | mouse CT-2A astrocytoma | SD-UR | 12-14 | 8 | 14.0 | 0.2 | 70.0 | Tumor dry weight: 95 ± 25 mg^{h} |
| | syngenic (C57BL/6J) | KD-UR | 12-14 | 8 | 13.5 | 0.6 | 22.5 | Tumor dry weight: 90 ± 15 mg |
| | | KD-R | 12-14 | 8 | 8.0 | 1.8 | 4.4 | Tumor dry weight: 35 ± 5 mg |
| | human U87 glioma | SD-UR | 12-14 | 8 | 11.5 | 0.5 | 23.0 | Tumor dry weight: 60 ± 10 mg^{h} |
| | xenograft (SCID) | KD-UR | 12-14 | 8 | 11.5 | 1.2 | 9.6 | Tumor dry weight: 60 ± 7 mg |
| | | KD-R | 12-14 | 8 | 5.5 | 3.0 | 1.8 | Tumor dry weight: 37 ± 5 mg |
| Mouse⁴⁵ | mouse GL261 astrocytoma | SD-UR | 19 | 13 | 10.0 | 0.2 | 50.0 | Median survival time: 23 days |
| | (C57BL/6-cBrd/cBrd/Cr) | KD-UR | 19 | 13 | 8.9 | 1.4 | 6.4 | Median survival time: 28 days |
| | | SD-UR + Rad^{e} | 11 | 13 | 9.7 | 0.3 | 32.3 | Median survival time: 41 days |
| | | KD-UR + Rad | 11 | 13 | 9.7 | 1.7 | 5.7 | Median survival time: 200+ days |

^{a}Ketogenic Diet, Unrestricted
^{b}Ketogenic Diet, Restricted
^{c}Standard Diet, Unrestricted
^{d}Standard Diet, Restricted
^{e}Diet with Radiation therapy
^{f}Blood/plasma beta-hydroxybutyrate measurement
^{g}Urinary ketones were measured
^{h}Mean ± 95% Confidence Interval

As shown in Table 1 above, the first clinical study evaluated two pediatric patients; one with an anaplastic astrocytoma, and another with a cerebellar astrocytoma (Nebeling L C, Miraldi F, Shurin S B, Lerner E. Effects of a ketogenic diet on tumor metabolism and nutritional status in pediatric oncology patients: two case reports. J Am Coll Nutr. 1995; 14:202-8). Both individuals were placed on a ketogenic diet for eight weeks. During the 8-week treatment period, GKI dropped from about 27.5 to about 0.7-1.1 in the patients. The patient with the anaplastic astrocytoma, who did not have a response to prior chemotherapy, had a 21.7% reduction in fluorodeoxyglucose uptake at the tumor site (no chemotherapy during diet). The patient with the cerebellar astrocytoma received standard chemotherapy concomitant with the ketogenic diet. Fluorodeoxyglucose uptake at the tumor site in this patient was reduced by 21.8%. Quality of life was markedly improved in both children after initiation of the KD (Nebeling L C, Miraldi F, Shurin S B, Lerner E. Effects of a ketogenic diet on tumor metabolism and nutritional status in pediatric oncology patients: two case reports. J Am Coll Nutr. 1995; 14:202-8).

The second clinical study evaluated a 65-yr.-old woman with glioblastoma multiforme (Zuccoli G, Marcello N, Pisanello A, Servadei F, Vaccaro S, Mukherjee P, et al. Metabolic management of glioblastoma multiforme using standard therapy together with a restricted ketogenic diet: Case Report. Nutr Metab (Lond). 2010; 7:33). The patient was placed on a calorie-restricted ketogenic diet (600 kcal/day) concomitant with standard chemotherapy and radiation, without dexamethasone, for eight weeks. The patient's GKI decreased from 37.5 to 1.4 in the first three weeks of the diet. No discernible brain tumor tissue was detected with MRI in the patient at the end of eight weeks of the calorie restricted ketogenic diet. It is also important to mention that the patient was free of symptoms while she adhered to the KD. Tumor recurrence occurred 10 weeks after suspension of the ketogenic diet.

The third study, a preclinical mouse study, evaluated the effects of diets on an orthotopically implanted CT-2A syngeneic mouse astrocytoma in C57BL/6 J mice (Seyfried T N, Sanderson T M, El-Abbadi M M, McGowan R, Mukherjee P. Role of glucose and ketone bodies in the metabolic control of experimental brain cancer. Br J Cancer. 2003; 89:1375-82). Mice were implanted with tumors and fed one of four diets for 13 days: 1) standard diet fed unrestricted, 2) calorie restricted standard diet, 3) ketogenic diet fed unrestricted, or 4) calorie restricted ketogenic diet. The mice fed a standard unrestricted diet and a ketogenic diet had rapid tumor growth after 13 days, and a GKI of 15.2 and 11.4, respectively. The group fed a calorie restricted standard diet had a significant decrease in tumor volume after 13 days, along with a GKI of 3.7. The group fed a calorie restricted ketogenic diet also had a significant decrease in tumor volume, along with a GKI of 4.4.

The fourth study evaluated the effects of diets on an orthotopically implanted CT-2A syngeneic mouse astrocytoma in C57BL/6 J mice and an orthotopically implanted human U87-MG human xenograft glioma in BALBc/6-severe combined immunodeficiency (SCID) mice (Zhou W, Mukherjee P, Kiebish M A, Markis W T, Mantis J G, Seyfried T N. The calorically restricted ketogenic diet, an effective alternative therapy for malignant brain cancer. Nutr Metab (Lond). 2007; 4:5). Tumors were implanted and grown in the mice for three days prior to diet initiation. After three days, mice were maintained on one of three diets for 8 days: 1) standard diet fed unrestricted, 2) ketogenic diet fed unrestricted, or 3) calorie restricted ketogenic diet. Tumor weights at the end of 8 days were reduced only in the mice that were fed a calorie restricted diet and experienced a significant decrease in GKI. Groups of mice that did not have a reduction in tumor weight had GKI's that ranged from 9.6-70.0. The groups of mice that had a reduction in tumor weight had GKI's that ranged from 1.8-4.4.

The fifth study evaluated the effects of diet and radiation on mouse GL261 glioma implanted intracranially in albino C57BL/6 J mice (Abdelwahab M G, Fenton K E, Preul M C, Rho J M, Lynch A, Stafford P, et al. The ketogenic diet is an effective adjuvant to radiation therapy for the treatment of malignant glioma. PLoS One. 2012; 7:e36197). The mice were implanted with tumors, and three days later they were placed on either a standard diet fed unrestricted or a ketogenic diet fed unrestricted. Mice were also assigned to groups that either received or did not receive concomitant radiation therapy. Without radiation, mice that were fed a ketogenic diet had a GKI of 6.4 and had a median survival of 28 days, compared to a GKI of 50.0 and median survival of 23 days for the standard diet group. With radiation, mice that were fed a ketogenic diet had a GKI of 5.7 and a median survival of 200+ days, compared to a GKI of 32.3 and median survival of 41 days for the standard diet group.

In addition to these studies, Table 2 shows a clear association of the GKI to the therapeutic action of calorie restriction against distal invasion, proliferation, and angiogenesis in the VM-M3 model of glioblastoma. The data for the GKI in Table 2 was computed from those mice that were measured for both glucose and ketones in comparison with the other biomarkers as previously described (Shelton L M, Huysentruyt L C, Mukherjee P, Seyfried T N. Calorie restriction as an anti-invasive therapy for malignant brain cancer in the VM mouse. ASN Neuro. 2010; 2:e00038). When viewed collectively, the results from the published reports show a clear relationship between the GKI and efficacy of metabolic therapy using either the KD or calorie restriction. Therapeutic efficacy of the KD or calorie restriction is greater with lower GKI values than with higher values. The results suggest that GKI levels that approach 1.0 are therapeutic for managing brain tumor growth. Further studies can determine those GKI values that can most accurately predict efficacy during metabolic therapy involving diet or procedures that lower glucose and elevate ketone bodies.

TABLE 2
Linking the Glucose Ketone Index (GKI) to the therapeutic action of calorie restriction against
distal invastion, proliferation, and angiogenesis in the VM-M3 model of glioblastoma.

| | Glucose | Ketone | | Distal invasion | Proliferation | Angiogenesis |
|---|---|---|---|---|---|---|
| Treatment | (mM) | (mM) | GKI | (photons/sec) | (KI [symbol] 7%) | (vessels/hpf) |
| AL | 11.2 ± 0.6 | 0.7 ± 0.09 | 15.3 ± 0.8 | 14 ± 1.3 | 4 [symbol] ± 1.2 | 15 ± 1.1 |
| CR | 83 ± 0.8 | 1.32 ± 0.1 | 6.5 ± 0.9 | 6 ± 0.9 | 34 ± 1.5 | 7 ± 0.72 |

AL, ad lib [symbol] feeding and CB is 60% food reduction for 10 days. Values are Mean ± SEM 3-7 mice were evaluated in each group: hpf, hight power [symbol] . [symbol]
indicates data missing or illegible when filed

The evidence shows that the GKI can predict success for brain cancer management in humans and mice using metabolic therapies that lower blood glucose and elevate blood ketone levels. Besides ketogenic diets, other dietary therapies, such as calorie restriction, low carbohydrate diets, and therapeutic fasting, can also lower blood glucose and elevate β-OHB levels and can have anti-tumor effects (Klement R J, Kammerer U. Is there a role for carbohydrate restriction in the treatment and prevention of cancer? Nutr Metab. 2011; 8:75; Fine E J, Segal-Isaacson C J, Feinman R D, Herszkopf S, Romano M C, Tomuta N, et al. Targeting insulin inhibition as a metabolic therapy in advanced cancer: a pilot safety and feasibility dietary trial in 10 patients. Nutrition. 2012; 28:1028-35; Klement R J. Calorie or carbohydrate restriction? The ketogenic diet as another option for supportive cancer treatment. Oncologist. 2013; 18:1056; Klement R J, Champ C E. Calories, carbohydrates, and cancer therapy with radiation: exploiting the five R's through dietary manipulation. Cancer Metastasis Rev. 2014; 33:217-29; Longo V D, Mattson M P. Fasting: molecular mechanisms and clinical applications. Cell Metab. 2014; 19:181-92; Raffaghello L, Safdie F, Bianchi G, Dorff T, Fontana L, Longo V D. Fasting and differential chemotherapy protection in patients. Cell Cycle. 2010; 9:4474-6; and Woolf E C, Scheck A C. The ketogenic diet for the treatment of malignant glioma. J Lipid Res. 2015; 56:5-10). The GKIC was developed to more reliably and simply predict therapeutic management for brain cancer patients under these dietary states than could measurements of either blood glucose or ketones alone. The data presented in Tables 1 and 2 support this prediction. The GKI concept has not been recognized or used previously to gauge success of various metabolic therapies based on inverse changes in glucose and ketone body metabolism.

As brain tumor cells are dependent on glucose for survival and cannot effectively use ketone bodies as an alternative fuel, a zone of metabolic management can be achieved under conditions of low glucose and elevated ketones. Ketone bodies also prevent neurological symptoms associated with hypoglycemia, such as neuroglycopenia, which allows blood glucose levels to be lowered even further (Veech R L, Chance B, Kashiwaya Y, Lardy H A, Cahill Jr G F. Ketone bodies, potential therapeutic uses. UBMB Life. 2001; 51:241-7 and Willemsen M A, Soorani-Lunsing R J, Pouwels E, Klepper J. Neuroglycopenia in normoglycaemic patients, and the potential benefit of ketosis. Diabet Med. 2003; 20:481-2). Hence, ketone body metabolism can protect normal brain cells under conditions that target tumor cells (Maalouf M, Rho J M, Mattson M P. The neuroprotective properties of calorie restriction, the ketogenic diet, and ketone bodies. Brain Res Rev. 2009; 59:293-315). The zone of metabolic management is considered the therapeutic state that places maximal metabolic stress on tumor cells while protecting the health and vitality of normal cells (Seyfried T N. Ketone strong: emerging evidence for a therapeutic role of ketone bodies in neurological and neurodegenerative diseases. J Lipid Res. 2014; 55:1815-17). Substantial data shows that the GKI is validated in several studies in mice. Prospective validation of the GKIC will be obtained from future studies using ketogenic diet therapy in humans with brain cancer and possibly other cancers that cannot effectively metabolize β-OHB for energy, and depend upon glucose for survival.

The GKI can be useful in determining the success of dietary therapies that shift glucose- and lactate-based metabolism to ketone-based metabolism. As a shift toward ketone-based metabolism underscores the utility of many dietary therapies in treating metabolic diseases (Meidenbauer J J, Ta N, Seyfried T N. Influence of a ketogenic diet, fish-oil, and calorie restriction on plasma metabolites and lipids in C57BL/6 J mice. Nutr Metab. 2014; 11:23 and Rieger J, Bahr O, Maurer G D, Hattingen E, Franz K, Brucker D, et al. ERGO: a pilot study of ketogenic diet in recurrent glioblastoma. Int J Oncol. 2014; 44:1843-52), the GKI can be used in determining the therapeutic success of shifting metabolism in individual patients. The GKI therefore can be used to study the effectiveness of dietary therapy in clinical trials of patients under a range of dietary conditions, with a composite primary endpoint consisting of lowering the subjects' GKI. This will allow investigators to parse the effects of successful dietary intervention on disease outcome from unsuccessful dietary intervention.

Recent clinical studies assessing the effects of dietary therapy on brain cancer progression have not measured both blood glucose and ketone bodies throughout the study periods (Rieger J, Bahr O, Maurer G D, Hattingen E, Franz K, Brucker D, et al. ERGO: a pilot study of ketogenic diet in recurrent glioblastoma. Int J Oncol. 2014; 44:1843-52. And Champ C E, Palmer J D, Volek J S, Werner-Wasik M, Andrews D W, Evans J J, et al. Targeting metabolism with a ketogenic diet during the treatment of glioblastoma multiforme. J Neurooncol. 2014; 117:125-31). Future clinical studies that intend to assess the effect of dietary therapy on brain tumor progression should measure both blood glucose and ketone, as these markers are necessary to connect dietary therapy to therapeutic efficacy. Preclinical studies have demonstrated a clear linkage between GKI and therapeutic efficacy. The GKI will be an important biomarker to measure in future rigorously designed and powered clinical studies in order to demonstrate if there is a linkage between GKI and therapeutic efficacy, as the few case reports in the literature suggest.

The zone of metabolic management is likely entered with GKI values between 1 and 2 for humans. Optimal management is predicted for values approaching 1.0, and blood glucose and ketone values should be measured 2-3 hours postprandial, twice a day if possible. This will allow individuals to connect their dietary intake to changes in their GKI. As an example, FIG. 2 uses the GKIC to track the GKI values of a hypothetical individual on a ketogenic diet, with a target GKI of 1.0. When an individual's GKI falls below the line denoting the target metabolic state, the zone of metabolic management is achieved. Further studies will be needed to establish the validity of the predicted zone of management.

The GKIC could have utility not only for managing brain cancer and possibly other cancers dependent on glucose and aerobic fermentation for survival, but also for managing other diseases or conditions where the ratio of glucose to ketone bodies could be therapeutic. Such diseases and conditions include Alzheimer's disease, Parkinson's disease, traumatic brain injury, chronic inflammatory disease, and epilepsy (Seyfried T N. Ketone strong: emerging evidence for a therapeutic role of ketone bodies in neurological and neurodegenerative diseases. J Lipid Res. 2014; 55:1815-17). For example, the ketogenic diet has long been recognized as an effective therapeutic strategy for managing refractory seizures in children (Freeman J M, Kossoff E H. Ketosis and the ketogenic diet, 2010: advances in treating epilepsy and other disorders. Adv Pediatr. 2010; 57:315-29 and Hartman A L, Vining E P. Clinical aspects of the ketogenic diet. Epilepsia. 2007; 48:31-42). Therapeutic success in managing generalized idiopathic epilepsy in epileptic EL mice can also be seen when applying the GKI to the data presented on glucose and β-OHB (Mantis J G, Centeno N A, Todorova M T, McGowan R, Seyfried T N. Management of multifactorial idiopathic epilepsy in EL mice with caloric restriction and the ketogenic diet: role of glucose and ketone bodies. Nutr Metab (Lond). 2004; 1:11). Healthy individuals can utilize the GKIC to prevent diseases and disorder, and manage general wellness. Further studies can corroborate the utility of the GKIC for predicting therapeutic success in the metabolic management of disease.

1. A method for managing the metabolic treatment of a subject comprising:
administering a diet to the subject, wherein the diet reduces blood glucose levels and elevates blood ketone levels in the subject;
tracking the ratio of blood glucose to blood ketone levels in the subject as a single Glucose Ketone Index value; and
maintaining the tracked Glucose Ketone Index value within a target range.

2. The method of claim 1, wherein the maintaining the tracked Glucose Ketone Index value within a target range comprises altering the diet administered to the subject.

3. The method of claim 1, wherein the diet comprises at least one of a ketogenic diet, calorie restricted diet, nutritional supplementation, pharmacological therapy, low carbohydrate diets, ketone ester supplementation, and therapeutic fasting.

4. The method of claim 1, wherein the target range is a Glucose Ketone Index value from about 0.5 to about 5.

5. The method of claim 1, wherein the metabolic treatment comprises treatment for a metabolic or inflammatory disease or indication.

6. The method of claim 5, wherein the metabolic disease or indication is a neurological or non-neurological disorder of metabolism.

7. The method of claim 6, wherein the neurological disorder is Alzheimer's disease, Parkinson's disease, traumatic brain injury, autism, migraines, or epilepsy.

8. The method of claim 5, wherein the inflammatory disease is a chronic inflammatory disease.

9. The method of claim 8, wherein the chronic inflammatory disease is obesity, Type 2 diabetes, or cardiovascular disease.

10. The method of claim 5, wherein the metabolic disease or indication is a cancer.

11. The method of claim 10, wherein the cancer is malignant brain tumors or other cancers that express aerobic fermentation known as the Warburg effect.

12. The method of claim 10, wherein the cancer is brain, breast, lung, or colon.

13. The method of claim 1, wherein the subject is an animal.

14. The method of claim 1, wherein the subject is a human.

15. The method of claim 1, wherein the blood Glucose Ketone Index is calculated from the presence of glucose and beta-hydroxybutyrate in the blood of the subject according to the formula
$$[ Glucose Ketone Index ] = \frac{[ Glucose ( mg / dL ) ] / 18.016}{[ β \text{-} OHB ( mM ) ]} .$$

16. The method of claim 1, wherein the blood Glucose Ketone Index is calculated from the presence of glucose and acetoacetate in the blood of the subject according to the formula
$$[ Glucose Ketone Index ] = \frac{[ Glucose ( mg / dL ) ] / 18.016}{2 * [ Acetoacetate ( mM ) ]} .$$

17. The method of claim 1, wherein the blood Glucose Ketone Index is calculated from the presence of glucose in the blood and acetone in the breath of the subject according to the formula
$$[ Glucose Ketone Index ] = \frac{[ Glucose ( mg / dL ) ] / 18.016}{0.11 * {[ Acetone ( mM ) ]}^{0.47} - 0.18} .$$

18. The method of claim 1, wherein the blood Glucose Ketone Index is calculated from the presence of glucose in the blood and urinary acetoacetate of the subject according to the formula
$$[ Glucose Ketone Index ] = \frac{[ Glucose ( mg / dL ) ] / 18.016}{0.45 * {[ Urinary Acetoacetate ( mM ) ]}^{0.5} + 0.1} .$$

19. The method of claim 1, wherein the tracking is performed by entering metabolic values into a Glucose Ketone Index Calculator.

20. The method of claim 1, wherein the target range is correlated to improved prognoses in a plurality of subjects in need of metabolic therapy.

21. The method of claim 1, wherein the Glucose Ketone Index value identifies a metabolic state of health.

22. The method of claim 1, wherein the Glucose Ketone Index target range is within a zone of metabolic management.

23. A device capable of computing the ratio of blood glucose to blood ketones as a single Glucose Ketone Index value.

24. The device of claim 23, wherein blood glucose values in units of mg/dL or mmol and blood ketone (β-OHB) levels in units of mg/dL or mmol are entered into the device and the units are converted to mg/dL or mmolar by one or more of the following equations:
$$\begin{matrix}[ Glucose Ketone Index ] = \frac{[ Glucose ( mg / dL ) ] / 18.016}{[ β \text{-} OHB ( mM ) ]} \\ [ Glucose ( mg / dL ) ] = [ Glucose ( mM ) ] \times 18.016 \\ [ Glucose ( mM ) ] = \frac{[ Glucose ( mg / dL ) ]}{18.016} \\ [ β \text{-} OHB ( mg / dL ) ] = [ β \text{-} OHB ( mM ) ] \times 10.41 \\ [ β \text{-} OHB ( mM ) ] = \frac{[ β \text{-} OHB ( mg / dL ) ]}{10.41} .\end{matrix}$$

25. The device of claim 23, wherein blood glucose values in units of mg/dL or mmol and blood ketone (acetoacetate) levels in units of mg/dL or mmol are entered into the device and the units are converted to mg/dL or mmol by one or more of the following equations:
$$\begin{matrix}[ Glucose Ketone Index ] = \frac{[ Glucose ( mg / dL ) ] / 18.016}{2 * [ Acetoacetate ( mM ) ]} \\ [ Glucose ( mg / dL ) ] = [ Glucose ( mM ) ] \times 18.016 \\ [ Glucose ( mM ) ] = \frac{[ Glucose ( mg / dL ) ]}{18.016} \\ [ Acetoacetate ( mg / dL ) ] = [ Acetoacetate ( mM ) ] \times 10.2088 \\ [ Acetoacetate ( mM ) ] = \frac{[ Acetoacetate ( mg / dL ) ]}{10.2088} .\end{matrix}$$

26. The device of claim 23, wherein blood glucose values in units of mg/dL or mmol and blood ketone (breath acetone) levels in units of mg/dL or mmol are entered into the device and the units are converted to mg/dL or mmol by one or more of the following equations:
$$\begin{matrix}[ Glucose Ketone Index ] = \frac{[ Glucose ( mg / dL ) ] / 18.016}{0.11 * {[ Acetone ( mM ) ]}^{0.47} - 0.18} \\ [ Glucose ( mg / dL ) ] = [ Glucose ( mM ) ] \times 18.016 \\ [ Glucose ( mM ) ] = \frac{[ Glucose ( mg / dL ) ]}{18.016} \\ [ Acetone ( mg / dL ) ] = [ Acetone ( mM ) ] \times 5.808 \\ [ Acetone ( mM ) ] = \frac{[ Acetone ( mg / dL ) ]}{5.808} .\end{matrix}$$

27. The device of claim 23, wherein blood glucose values in units of mg/dL or mmol and blood ketone (urinary acetoacetate) levels in units of mg/dL or mmol are entered into the device and the units are converted to mg/dL or mmol by one or more of the following equations:
$$\begin{matrix}[ Glucose Ketone Index ] = \frac{[ Glucose ( mg / dL ) ] / 18.016}{0.45 * {[ Urinary Acetoacetate ( mM ) ]}^{0.5} + 0.1} \\ [ Glucose ( mg / dL ) ] = [ Glucose ( mM ) ] \times 18.016 \\ [ Glucose ( mM ) ] = \frac{[ Glucose ( mg / dL ) ]}{18.016} \\ [ Urinary Acetoacetate ( mg / dL ) ] = [ Urinary Acetoacetate ( mM ) ] \times 10.2088 \\ [ Urinary Acetoacetate ( mM ) ] = \frac{[ Urinary Acetoacetate ( mg / dL ) ]}{10.2088} .\end{matrix}$$
